(19) United States
(12) Patent Application Publication
(10) Pub. No.: US 2006/0067467 A1
(43) Pub. Date: Mar. 30, 2006

(54) Apparatus and method for conformal radiation brachytherapy for breast and other tumors

(75) Inventors: Victor I. Chornenky, Minnetonka, MN (US); Ali Jaafar, Eden Prairie, MN (US)

Correspondence Address: OFFICES OF CRAIG GREGERSEN, P.O. BOX 386353, 10032 QUEBEC AVENUE SOUTH, BLOOMINGTON, MN, 55438, US

(73) Assignee: Minnesota Medical Physics LLC

(21) Appl. No.: 11/236,147

(22) Filed: Sep. 27, 2005

Related U.S. Application Data
(60) Provisional application No. 60/613,210, filed on Sep. 28, 2004.

(51) Int. Cl. A61N 5/10

(52) U.S. Cl. 378/65

(57) ABSTRACT

A system and method providing conformal x-ray brachytherapy for treatment of a body having a tumor by irradiation of a target volume of tissue in a patient is disclosed wherein an x-ray probe including an x-ray emitter, an imaging probe configured to image the target volume, a translation stage mounting the x-ray probe for translational motion, a rotation stage mounting the x-ray probe for rotational motion, a compliant balloon inserted into a cavity created in a body by excision of a tumor, and computer operatively connected to the x-ray and imaging probes and the rotation and translation stages are provided to image and control the operation of the x-ray probe to irradiate the target volume according to predetermined treatment protocols.

### BACKGROUND OF THE INVENTION

This application claims priority from U.S. Provisional Patent Application Ser. No. 60/613,210 entitled Apparatus And Method For Conformal Radiation Brachytherapy, filed Sep. 28, 2004 and from U.S. patent application Ser. No. 10/392,167 (Published Application No. 20030179854) entitled “X-Ray Apparatus With Field Emission Current Stabilization And Method Of Providing X-Ray Radiation Therapy” and filed on Mar. 19, 2003 and U.S. patent application Ser. No. 10/938,971 (Published Application No. 20050038488), also entitled “X-Ray Apparatus With Field Emission Current Stabilization And Method Of Providing X-Ray Radiation Therapy” and filed on Sep. 10, 2004.

Radiation therapy has been and will for the foreseeable future continue to be an available and oft-used treatment modality (either alone or in some combination with surgery, chemotherapy, and/or hormone therapy) for the occurrence of cancerous tumors. Examples of the types of tumors treated with radiation therapy include cancers of the prostate, the breast, the lung, and the brain, head and neck, amongst others. Typically, the radiation therapy is provided to a localized tissue area surrounding the tumor. Depending upon the type of tumor and its location, the tumor may be excised prior to radiation therapy or it may be left in place and treated with radiation also.

Broadly speaking, treatment of a body with radiation because of such tumors can occur with the use of either internal (also known as brachytherapy) or external radiation sources. Both internal and external radiation sources have their own respective advantages and disadvantages well known to practitioners. Generally in external radiation therapy, a plurality of angles of exposure are used to irradiate the tumor and/or the surrounding marginal tissue so as to provide overlapping coverage of the tumor. The effect of the overlapping coverage is to ensure that the largest radiation dose is received at the desired treatment location while minimizing the radiation damage to the surrounding tissue. For example, typical slow-growth prostate gland tumors are typically not excised prior to radiation therapy. When treating such tumors with radiation, care should be taken to avoid or minimize radiation damage to the urethra, the rectum, and the peripheral nerve bundle of the prostate gland. Damage to the latter could lead to impotence. Yet, effective treatment requires that sufficient radiation be delivered to the prostate gland to destroy the cancerous cells. As another example, breast cancers are typically excised and the margin tissue surrounding the excised tumor is treated with radiation to hopefully kill any remaining cancer cells. Were this tissue to be treated externally from a single angle, radiation burns along the beam path would almost surely result in unwanted and undesirable damage to healthy tissue.

Thus, a common element in the successful use of either an internal or external radiation source for therapy that also minimizes radiation damage is knowledge of the geometry of the desired treatment volume. Knowing the geometry of the desired treatment volume, with or without tissue excision, enables the therapist to target that treatment volume from multiple angles and to reduce thereby the exposure of surrounding tissue to radiation.

More specifically, in the last 10-15 years, a new technology in radiation therapy has improved targeting accuracy, thereby allowing higher, more effective doses to be delivered to a tumor bed while minimizing side effects and complications. This new modality of therapy uses multiple specially shaped or “modulated” beams applied from several different directions to the target volume—that volume of tissue including the tumor and surrounding tissue to be target for receipt of therapeutic x-ray radiation. The main objective of the therapy is to concentrate radiation on tumors and minimize radiation dosages applied to the adjacent healthy tissue, especially to the critical parts of the body that are more sensitive to radiation. This technology is called Intensity-Modulated Radiation Therapy (IMRT), an advanced form of external beam irradiation that is commonly referred to as three-dimensional conformal radiation therapy (3DCRT).

Several advances in medical technology made the 3DCRT possible. The most important one was the development of sophisticated 3D imaging techniques, among them computer-assisted tomography (CAT), magnetic resonance imaging (MRI), ultrasound (US), and positron emission tomography (PET).

Each of the aforementioned imaging technologies utilize different tissue properties to distinguish adjacent tissues from each other. For example, CAT scans, MRI and US use physical properties of tissues to distinguish one tissue from another while PET scans utilize metabolic differences between malignant and healthy tissues. More specifically, CAT scans utilize differences in the various tissue electron densities to distinguish one tissue type from another. MRI uses differences in the hydrogen densities of various tissues to distinguish one from the other. Ultrasound imaging, on the other hand, uses differences in the acoustic properties of tumors and surrounding tissues, which results in reflections of ultrasound waves at the boundary of two tissues having different sound transmission speeds.

Development of the CAT scans enabled three-dimensional reconstructions of a patient's anatomy with high spatial resolution. This imaging modality provides substantially better visualization of the cancer and surrounding normal tissue in three dimensions. With this comprehensive ability to identify the target volume and the surrounding normal tissues in three-dimensional space, physicians can customize the shapes of radiation beams for each patient and more precisely aim a beam into the target volume from multiple directions while substantially reducing the exposure of surrounding normal tissues to the radiation beams.

Another important modality of 3D imaging that has been significantly improved over the last decade is MRI. MRI allows better differentiation between malignant and healthy tissues and is known for providing sharp differentiations between tumors and surrounding soft tissues, for example in the brain or prostate gland. As its resolution continues to improve, MRI becomes increasingly involved in cancer diagnosis and therapy.

All these imaging modality give somewhat different 3D images of the gross tumors and disseminated micro tumors around them. They compliment each other; combined together they allow a diagnostician to compile a better diagnostic image of the tumor bed and thereby enable the physician to delineate the target volume and adjacent critical structures more precisely.

Another imaging advance is the ability to rotate an image of a patient's anatomy in 3D virtual space and, especially newly developed software called Room's-Eye-View (REV). This functionality gives radiation oncologists a tool for customizing radiation beam cross sections and directions for irradiation of the tumor that provide high conformity with an identified 3D target volume. This software tool provides an interactive three-dimensional isodose surface display, which is a valuable tool for evaluation of proposed 3D radiation therapy dose distributions in terms of ensuring adequate coverage of the target volume while sparing critical structures. The REV display enables radiation oncologists to view a target volume or a normal tissue volume with superimposed isodose surfaces or “dose clouds” from any arbitrary viewing angle. Using different multi-leaf collimators to shape the radiation beams generated by therapeutic machines oncologists have succeeded in increasing doses for malignant tumors and sparing critical structures around them thus improving the local control of the disease and decreasing toxicity not only for critical structures but for the adjacent tissues in general.

Another approach for conformal radiation therapy has been developed wherein brachytherapy is provided by implantation of radioactive seeds that covering the target volume with the desired radiation dose. This therapy modality It uses real time computation of the 3D distribution of the radiation dose received by the target volume and surrounding tissue as the oncologist places the seeds.

To achieve high quality radiation therapy, it is necessary to accurately relate the positions of target volumes and critical structures in the patient to the positions and orientation of beams used for imaging and treatment. This requires the use of multiple coordinate systems, one within the patient and those related to the imaging and treatment machines. The positions of target volumes and critical structures are related to anatomic reference points or alignment marks in the coordinate system of the patient. The position and orientation of the imaging and treatment machines are defined in the coordinate systems related to these machines. Because the reference points of the patient's anatomy and special radio opaque marks made on the patient skin can be defined in both patient and machine coordinate system, they can serve as a link between these two systems thus allowing the coordinates of the target volumes and critical structures to be defined relative to the treatment machine for treatment planning and the actual radiation treatment.

Another significant advance in the conformal technique is the use of electron accelerators for radiation treatment as compared to the high photon energy x-ray machines. The advantage of the several megavolts electron beam is that it deposits the ionizing energy preferentially at some predetermined depth in the tissue, thus sparing the skin and increasing the dose in the tumor.

The primary achievement of conformal therapy is a better local control of the disease that translates into longer survival rate of the patients. This better control is achieved by raising the radiation dose received by a tumor up to 80 Grays (Gy) while reducing injury to the critical structure around the tumor.

Drawbacks of the external beam conformal radiation therapy are that it is a time consuming and expensive modality of radiation treatment. In addition, there is some significant room for improvement of the procedure and apparatus.

One complication in the use of either external beams or brachytherapy radiation is that the present systems for radiation therapy tend to depend somewhat if not heavily on the existence of a symmetric treatment volume, though symmetrical tumors are less common than asymmetrical tumors. For example, with some tumors, such as lumps in the breast, the geometry of the cavity left by following excision of the tumor is asymmetric. That is, rarely does a breast tumor take the form of a perfect or near perfect sphere. When the tumor and surrounding margin tissue is excised then, either an irregularly configured cavity is left or the surgeon is forced to remove supposed otherwise healthy tissue in order to produce a more symmetric cavity. The irregular shape of the cavity left after excision makes it difficult for external beam sources to provide the desired dose within the target volume. Certain brachytherapy systems can treat somewhat irregularly configured cavities, but only by inflating an non-compliant device within the cavity to create a nearly symmetrical surface, resulting in some tissue surrounding the device being stretched and other tissue being compressed, thereby affecting the received dose in each portion of the target volume.

An object of the current invention is to improve the quality of the conformal therapy and reduce cost of the radiation treatment.

Another object is to provide a highly automated high dose rate x-ray brachytherapy system.

Another object is to provide a radiation therapy system wherein the ionizing radiation comprises low energy x-rays in the range of energies 10-50 keV. Low energy x-rays provide very high gradients of the delivered dose, which can be instrumental in sparing the critical structures.

Another object is to provide better protection for medical personnel that perform radiation treatment. Low energy x-ray systems of the type contemplated for use in accord with the present invention do not require expensive bunker type radiation treatment facilities such as is required with radiation sources such as radioactive sources. Thus, it is easier to protect medical personnel from unnecessary and damaging radiation exposure when performing a procedure using the apparatus and method of the present invention.

Another particular object is to avoid extensive irradiation of the remaining breast tissue where a breast is being treated after detection of lumps.

Another object of the present invention is to provide a system and method for treatment of asymmetric tumors as well as symmetric tumors.

### BRIEF DESCRIPTION OF THE INVENTION

The present invention provides apparatus and method for providing three dimensional conformal radiation therapy that enables a therapist to deliver a desired radiation dose to a target volume while reducing exposure of the surrounding tissue and critical structures. In one aspect of the present invention there will be provided an x-ray probe having proximal and distal ends and an x-ray emitter disposed at the distal end. The probe is mounted for translational and rotational motion relative to a compliant balloon that is disposed within the target volume. The inner surface of the compliant balloon defines an isodose surface. The balloon includes an axial hollow shaft configured to receive the x-ray probe and, if desired, an imaging tool such as an ultrasound probe or a laser. The isodose surface is imaged either internally with the laser or the ultrasound probe or externally with an ultrasound probe, MRI, or other preferred imaging technology. In operation the imaging tool is used to image the isodose surface to identify a three-dimensional surface. The geometry of the three dimensional isodose surface is transferred to a computer, which uses the information to define the dwelling times and the linear and rotational motions of the x-ray probe so as to provide the desired radiation dose at the inner surface of the compliant balloon, and hence the identified target volume.

In one aspect of the present invention the x-ray and imaging probes are operatively connected to a computer including a memory that stores the identified target volume as well as radiation dose parameters. Appropriate software within the computer will adjust the translational position of the x-ray probe to a first desired irradiation position and the x-ray emitter will be activated to deliver a desired irradiation dose at a first location relative to the target volume/prostate gland. Preferably the x-ray emitter will have a narrow beam emission, enabling precise regions of the target volume to be targeted. The emitter can be rotated to sweep out a desired treatment volume and repositioned translationally. Dwelling times at each translational and rotational position will be determined prior to operation to ensure appropriate radiation dosages are received by the target volume while minimizing exposure of surrounding and critical tissues to the radiation.

In another aspect of the present invention a method of treating a tumor is provided. A tumor and surrounding margin tissue are identified using one or more of CAT scans, MRI, PET, ultrasound or other appropriate technologies. The tumor is excised and a compliant balloon is disposed within the resulting cavity. A target volume for treatment and a treatment regimen are determined including one or more locations for positioning an elongate x-ray probe having an x-ray emitter at its distal end relative to the target volume. The x-ray probe is disposed within the shaft of the compliant balloon and is moved translationally and rotationally to provide a predetermined therapeutic radiation dose to the target volume.

The foregoing objects and features of the present invention, as well as other various features and advantages, will become evident to those skilled in the art when the following description of the invention is read in conjunction with the accompanying drawings as briefly described below and the appended claims. Throughout the drawings, like numerals refer to similar or identical parts.

### BRIEF DESCRIPTION OF THE DRAWINGS

FIG. 1 illustrates a breast in a schematic side view showing an asymmetric tumor, the excision boundary, and a treatment volume.

FIG. 2 illustrates the breast of FIG. 1 after excision of the tumor.

FIG. 3 illustrates the breast of the FIG. 1 with a compliant balloon disposed within the cavity left by the tumor excision

FIG. 4 illustrates an embodiment of the present invention useful in treating a breast having a tumor that has been excised.

### DETAILED DESCRIPTION OF THE INVENTION

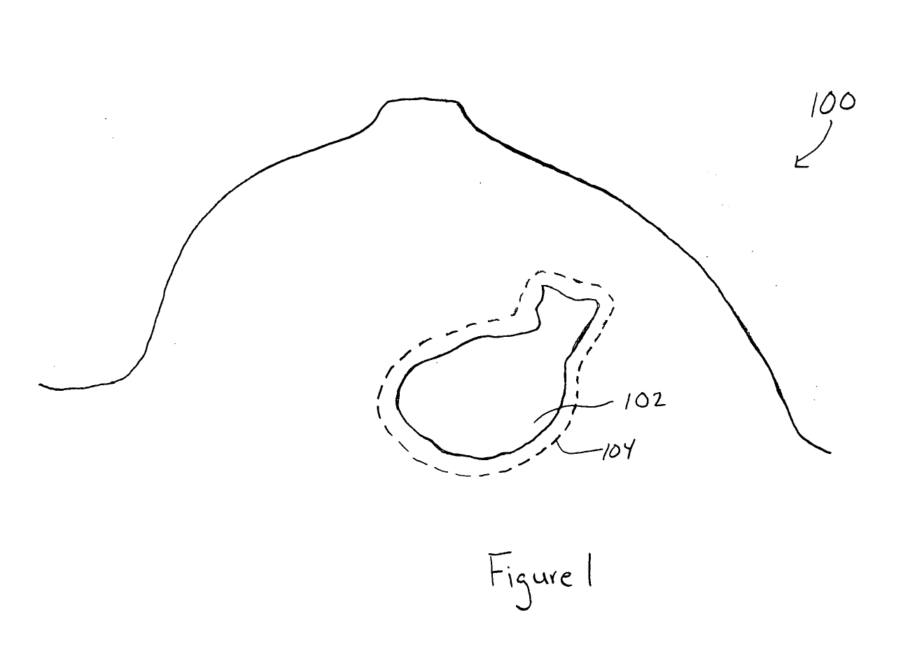

FIG. 1 illustrates in two dimensions a breast 100 having a tumor 102. Tumor 102 will be excised by a surgeon along with breast tissue surrounding the tumor to the extent indicated by dashed line 104. It will be understood that the Figure is meant to be representational only and the actual tumor and the excision boundary will define 3 dimensional shapes.

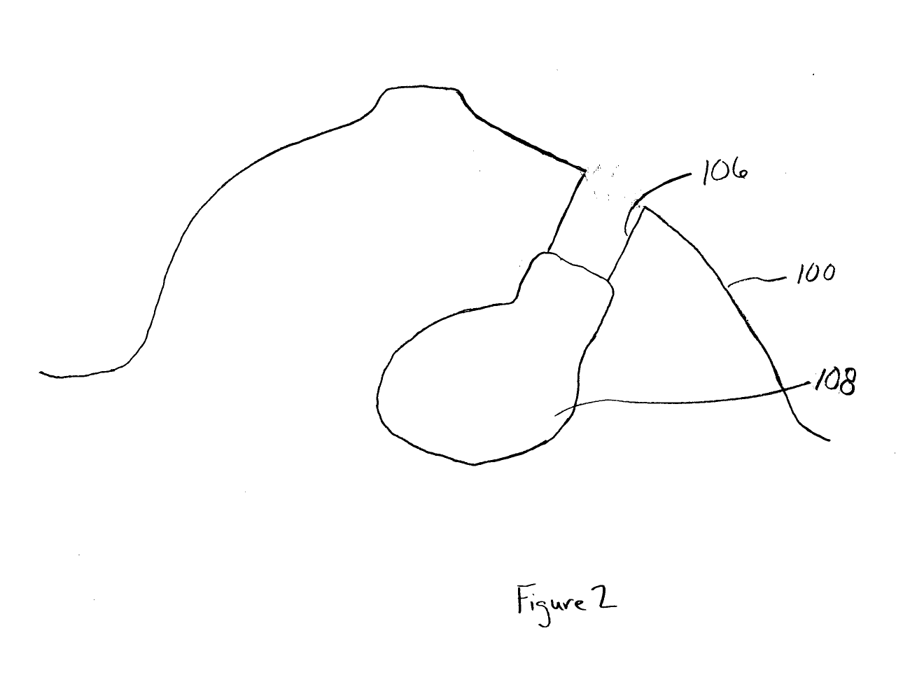

FIG. 2 illustrates the breast 100 of FIG. 1 after excision of the tumor through an incision 106, resulting in a cavity 108. Margin tissue surround a tumor is removed in an attempt to kill as many as possible of the cancerous cells that exist in the patient in an attempt to arrest further development of the disease. Often times the safest course of action is removal of the entire breast in a procedure known as a mastectomy. Due to the severe disfigurement such a procedure causes as well as because of the accompanying psychological trauma and subsequent therapeutic treatments, such as chemotherapy and/or radiation, many patients choose a lumpectomy instead and accompanying chemotherapy and/or radiation therapy.

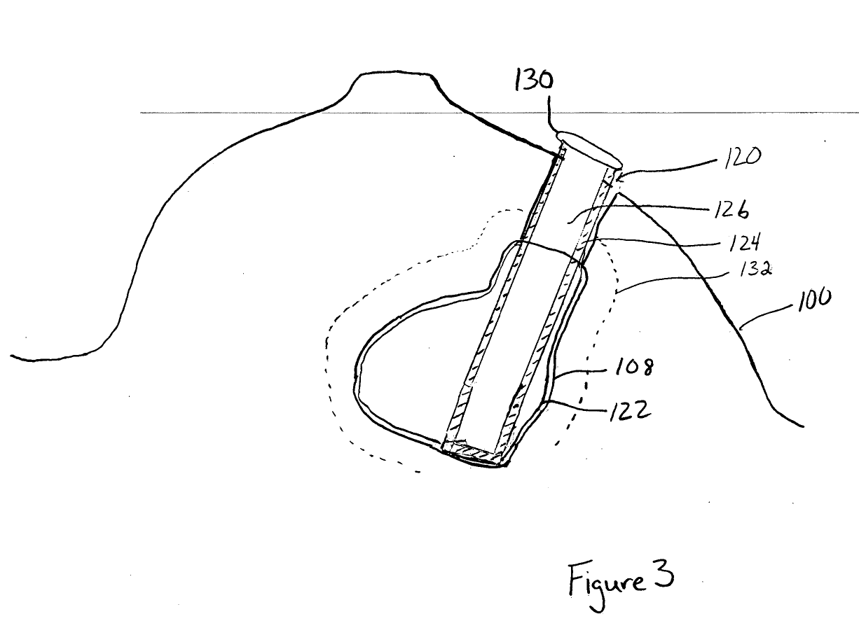

Referring now to FIG. 3, a compliant balloon 120 has been placed within the cavity 106 and inflated such that the compliant skin 122 of the balloon 120 bears against the inner wall of the cavity 108 and incision 106 when inflated in any known manner, including saline or air, the details of which are well known and have been omitted for clarity. The balloon 120 includes a central, elongate, hollow shaft 124 having any desirable and appropriate cross-sectional shape. Shaft 124 defines a central passage 126 and is configured and sized to receive therein the apparatus hereafter described. The compliant balloon skin 122 should be of a material that will allow it to inflate under the pressure of the inflating fluid, whether air or liquid, and substantially conform to the shape of the cavity 108, thus defining an isodose surface. Balloon 120 may include an attachment collar 130 appropriately configured to mate with an x-ray or imaging probe described hereafter. As is well known, many such configurations are known and acceptable for such use and hence are not illustrated herein.

FIG. 3 also illustrates the proposed treatment volume 132 surrounding the cavity 108. The treatment volume 132 is identified by the surgeon as a predetermined depth of tissue surrounding the cavity 108. The goal of irradiating the target volume is the hope that all remaining cancer cells within the patient's breast and body will be killed and thus that the disease progression will be terminated. As with many, if not the large majority, of such treatment or target volumes do not form symmetrical structures. Irradiation of the target volume is thus problematic at best using an external beam source since so many beam angles would be necessary to ensure overlapping coverage of the target volume. A large number of irradiation angles, of course, increases the risk of radiation damage to healthy tissue whose preservation is desired. The use of radioactive sources to irradiate the target volume internally is also problematic since precise control of the dose in any one direction is difficult to control, particularly where the cavity has an irregular or asymmetric configuration. For this reason, amongst others, non-compliant balloons are used to provide a defined symmetrical shape by stretching the cavity into a predetermined configuration surrounding the non-compliant or rigid balloon structure.

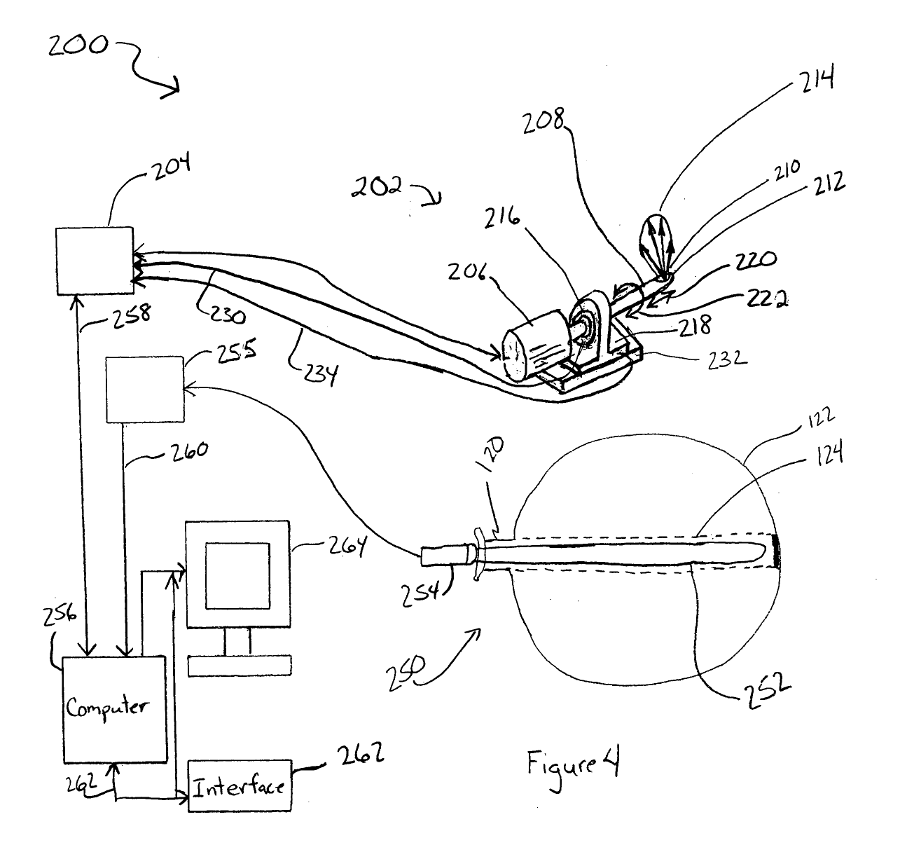

The system of the present invention addresses the concerns over radiation therapy for such asymmetrically configured tumors and target volumes. Thus, referring to FIG. 4, a system 200 for conformal radiation brachytherapy of a patient following tumor excision is illustrated. System 200 will be shown and described relative to therapeutic x-ray treatment of the breast of a human female, though its use relative to other tumors will be understood.

System 200 comprises a therapeutic x-ray unit 202 including a controller 204, a vacuum housing 206, and an elongated hollow probe 208 connected to the vacuum housing 206. Probe 208 has an x-ray emitter 210 at its distal tip 212 generating a directional x-ray side beam 214. The elongated probe 208 of the x-ray unit 202 is secured to a rotational stage 216 that may be, in its turn, connected to a linear stage 218 that during operation provides translational or longitudinal motion of the elongated probe (and the x-ray emitter 210 at its tip) along the probe axis as indicated by double-headed arrow 220. The rotational stage 216 during operation of the system provides rotational motion of the x-ray emitter and its side beam 214 around the axis of the elongated probe 208 as indicated by double-headed arrow 222.

Rotational stage 216 communicates with the x-ray controller 204 via an appropriate connector 230 providing the controller with angular coordinates of the emitter beam 210 and receiving commands for further execution of the rotational motion. The linear stage 218 may rest on a steady base 232. Linear stage 218 communicates with the x-ray controller 204 via an appropriate connector 234. Linear stage 218 provides translational or longitudinal coordinates of the x-ray beam and receives commands from the controller 204 about succeeding motions and dwelling times.

System 200 also includes an imaging probe. Such a probe could take the form of an ultrasound or laser probe. FIG. 4 illustrates an ultrasound imager in accord with the present invention. Thus, system 200 includes an ultrasound imaging system 250 comprising an imaging probe 252. Probe 252 is shown disposed within shaft passage 126 of balloon 120 to illustrate an internal scanning operation, though as will be explained later the probe 252 could also be used externally to produce an image. Probe 252 may include an electromechanical block 254 that provides longitudinal and angular positioning of the ultrasound probe 252. Imaging system 250 will also include an ultrasound imaging unit 255 supplying a computer 256 with ultrasound imaging data and a display 264 providing image 2D slices and 3D imaging of the target volume 130. The ultrasound probe 252 may be positioned within the passage 126 of balloon shaft 124. In that position, the probe can be rotated and translated as desired to provide a three dimensional image of the cavity 104. When used in such a manner, the balloon itself can include the necessary fiducials to map out the target volume 132. Alternatively, the probe 252, though shown as an elongated probe configured to be used in conjunction with the balloon 120, other configurations suitable for an external imaging of the balloon 120, and hence the target volume 132, can be used also in the present system.

X-ray controller 204 communicates with the system computer 256 via an appropriate connector 258 while the ultrasound imaging unit 255 communicates with the same computer via an appropriate connector 260. Computer interface 262, which may be a keyboard or any other useful interface that enables control of the system 200 by an operator, is connected to the system computer 256 via an appropriate connector 262. A display 264 is connected to the computer 256 via an appropriate connector 266.

Rather than an ultrasound imager, system 200 may include an elongate probe-like laser imager configured to be received within the shaft passage 126. Such a probe may be disposed within the passage 126 and operated so as to cause a laser beam to emanate therefrom and reflect from the inner surface of balloon 120, thus enabling a three dimensional image of the inflated balloon skin to be constructed, and hence the target volume to be irradiated.

In operation, the target volume will be identified in a patient by imaging the balloon internally with a laser imager or an ultrasound imager or externally with an ultrasound imager or any other imaging system capable of providing a three dimensional image of the intended target volume utilizing other known or future medical imaging technologies. The coordinates of the target volume will be identified relative to the balloon 120. Information regarding the target volume, including its coordinates and treatment protocols (dose rate, total dose, position, dwelling time at any one position, etc.) will be provided to the computer 256. Once imaging is complete, the imager will no longer be necessary for the procedure. The x-ray probe 202 will be operationally placed relative to the target volume by inserting the probe into the shaft passage 126 of balloon 120 after the internal imaging probe has been removed, if used in lieu of an external imager, and attached thereto for the procedure. Because the target volume has been identified relative to coordinates based upon the balloon, the operating parameters of the probe 202 can also be related thereto. The x-ray probe 202 will be attached to the balloon 120 in any known manner so as to provide precise rotational and translational movement thereof.

The probe location can be adjusted translationally and rotationally and operated so as to provide the desired x-ray radiation therapy at the desired dose levels to the target volume. As can be seen from FIGS. 2 and 3, the cavity surface will vary angularly and in distance from the shaft 124 of the balloon 120. By providing rotation and translation of the x-ray probe 202 as well as the intensity of the beam 214, the operator can deliver precise levels of radiation to all areas of the target volume 132 while minimizing the exposure of the surrounding tissue to radiation. The present system, in particular its ability to deliver precise radiation doses to precise locations inside the target volume, thus aids in the maximization of the benefits of radiation therapy while minimizing its side effects.

Stated otherwise, it will be understood that the system 100 disclosed and discussed herein can be utilized to position the x-ray probe in a plurality of locations relative to the target volume 132, thus providing the therapist with the ability to irradiate the target volume from multiple directions and at multiple x-ray strengths so as to precisely tailor the therapy to provide the maximum dose to the target volume and reduced dosages to the tissues lying outside the target volume. The exact treatment protocols will be selected in the diagnostic stage to maximize the therapeutic effects of the therapy.

The algorithm defining the administration of radiation therapy may include known parameters of the beam 214, such as the direction in 3D space of the beam, the dose rate, and a radial function describing decreasing the dose rate with radial distance due to absorption in tissue (depth of penetration), which in its turn is defined by the operating voltage of the x-ray emitter. The algorithm selects dwelling times for the x-ray beam with a given angular and linear coordinates to deliver to the 3D surface contouring the treatment volume a predetermined dose.

The present invention has been described in language more or less specific as to the apparatus and method features. It is to be understood, however, that the present invention is not limited to the specific features described, since the apparatus and method herein disclosed comprise exemplary forms of putting the present invention into effect. For example, while an ultrasound probe has been illustrated as being the operational, real-time imaging apparatus during a therapeutic procedure, other compact imaging devices may appear in the near future and such would also be usable in accord with the present invention provided such use would be within acceptable safety considerations for a therapeutic procedure. In addition, while the invention has been described relative to its use in treatment of an asymmetrically configured tumor found in a human female breast, it will be understood that the invention could also be used for symmetrically configured tumors as well, and in locations other than the breast or in non-human patients. The invention is, therefore, claimed in any of its forms or modifications within the proper scope of the appended claims appropriately interpreted in accordance with the doctrine of equivalency and other applicable judicial doctrines.

What is claimed is:

1. A system providing conformal x-ray brachytherapy for treatment of a tumor by irradiation of a target volume of tissue in a patient after excision of the tumor, said system comprising:
an inflatable, compliant balloon disposed within the cavity created by excision of the tumor, said balloon including a hollow shaft defining a passage therein and a compliant skin;
an x-ray probe including an x-ray emitter, said probe configured to be received within said passage;
an imaging probe configured to image the target volume;
a translation stage mounting said x-ray probe for translational motion;
a rotation stage mounting said x-ray probe for rotational motion; and
a computer operatively connected to said x-ray and imaging probes and said rotation and translation stages, said computer directing translational and rotational motion of said x-ray probe.

2. The system of claim 1 wherein the tumor is located in a breast.

3. The system of claim 1 wherein said x-ray probe is elongated and configured for insertion into said shaft.

4. The system of claim 1 wherein said imaging probe is configured for insertion into said shaft.

5. The system of claim 4 wherein said imaging probe is an ultrasound probe.

6. The system of claim 4 wherein said imaging probe is a laser probe.

7. The system of claim wherein said x-ray probe emits a side directional x-ray beam.

8. The system of claim 1 wherein said imaging probe is an ultrasound probe.

9. The system of claim 1 wherein said imaging probe is a laser.

10. A method providing conformal x-ray brachytherapy for treatment of a tumor in a patient by irradiation of a target volume of tissue in a patient, said method comprising:
excising the tumor to create a cavity within the patient;
disposing an inflatable balloon having a compliant skin and a shaft having a hollow passage within the cavity;
inflating the balloon;
providing an x-ray probe configured to be received within the passage and including an x-ray emitter translationally and rotationally movable;
providing a computer for controlling the translational and rotational position of the x-ray probe during a procedure and the radiation dose emitted by the x-ray probe;
disposing the x-ray probe in proximity of the target volume; and
irradiating the target volume according to predetermined therapeutic protocols.

11. The method of claim 10 wherein the x-ray probe emits a narrow emission beam.

12. The method of claim 10 and further including imaging the balloon with an imaging probe to define the target volume.

13. The method of claim 12 wherein the imaging probe is an ultrasound probe.

14. The method of claim 12 wherein the imaging probe is a laser.

15. The method of claim 10 wherein the tumor is in a breast.

16. The method of claim 10 and further including translating and rotating the x-ray probe to multiple spatial locations to provide irradiation of the target volume.
